(19) United States
(12) Patent Application Publication
(10) Pub. No.: US 2022/0261706 A1
(43) Pub. Date: Aug. 18, 2022

(54) SYSTEM AND METHOD FOR BOOKING APPOINTMENTS WITH HEALTH CARE PROFESSIONALS

(71) Applicant: VirtualMD Corporation, Toronto (CA)

(72) Inventor: Baldeep Takhar, Kitchener (CA)

(21) Appl. No.: 17/668,896

(22) Filed: Feb. 10, 2022

Related U.S. Application Data
(60) Provisional application No. 63/148,678, filed on Feb. 12, 2021.

(51) Int. Cl. G06Q 10/02; G06Q 10/10; G16H 40/20

(52) U.S. Cl. CPC G06Q 10/02; G16H 40/20; G06Q 10/1095

(57) ABSTRACT

A booking system including a processor and a data storage device communicatively coupled to the processor. The data storage device stores a patient profile and a schedule associated with a doctor. The processor is operable to receive, via a navigator style interface on a patient electronic communication device, a response requesting an urgent care appointment, identify and book for the patient a next-available appointment slot in the schedule of the doctor on the data storage device, send the patient's profile to a doctor electronic communication device of the doctor, and present the first patient with a set of details of the next-available appointment including a time at which the doctor will call the patient.

### CROSS-REFERENCE TO RELATED APPLICATION

This application claims the benefit of U.S. Provisional Patent Application No. 63/148,678, filed Feb. 12, 2021 and the entire content of U.S. Provisional Patent Application No. 63/148,678 is hereby incorporated by reference.

### TECHNICAL FIELD

The disclosure relates to appointment booking, and in particular to booking appointments with health care professionals.

### BACKGROUND

Many medical appointments do not require a physical face-to-face meeting between a doctor and a patient. Often, if the patient is able to accurately describe symptoms and respond to questions, the health care professional is able to diagnose a condition or recommend a course of action without physically examining the patient.

Even if the health care professional needs to visually inspect the patient to be able to confidently recommend a course of action, the health care professional can often receive the required visional information via a video from the patient rather than a physical exam of the patient. For example, the patient can use a smartphone with a camera to take one or more image of the patient's body and send the images to the doctor to review. In other cases, the patient may be able to send physical samples that the doctor needs for the diagnosis to the doctor or a third party to review, without the patient actually meeting the doctor in person.

This type of ‘virtual’ appointment with a health care professional can be arranged in the conventional manner. Many health care professionals have a receptionist handling the scheduling of appointments for the health care professional. Setting up an appointment with the health care professional involves a call to the receptionist. The receptionist will typically ask the patient for personal information, such as a name or other identifier, and will then check to see is the patient is eligible to book an appointment. The receptionist will then view a calendar for the health care professional and discuss options with the patient over the phone until an acceptable time and date can be found and booked for the patient.

A receptionist may be able to book virtual appointments between health care professionals and patients. However, there remains a need for a more versatile booking system.

### SUMMARY

According to some aspects, there is provided a booking system, comprising (a) at least one processor; and (b) at least one data storage device communicatively coupled to the at least one processor, and (c) wherein the at least one processor is operable to: (i) receive a referral request from a family doctor electronic communication device of a family doctor requesting to refer a patient to a specialist, the referral request including at least one criteria, (ii) identify a plurality of specialist doctor profiles stored on the at least one data storage device and matching the at least one criteria, and (iii) send a specialist notification to the family doctor electronic communication device for each of the plurality of specialist doctor profiles, each specialist notification including information from the associated specialist doctor profile.

In some examples, the at least one processor is further configured to (i) receive, in response to sending the notifications, a selection from the family doctor electronic communication device of a first specialist doctor profile of the plurality of specialist doctor profiles; and (ii) send a patient notice to a specialist doctor electronic communication device associated with the first specialist doctor profile, the patient notice including patient information from a patient profile of the patient stored on the at least one data storage device.

The at least one processor may be further configured to generate a referral notice and present the referral notice to the patient, wherein the referral notice includes information from the first specialist doctor profile.

According to some aspects, there is provided a booking system, comprising (a) at least one processor; and (b) at least one data storage device communicatively coupled to the at least one processor, and (c) wherein the at least one processor is operable to: (i) receive a request from a patient electronic communication device of a patient for an urgent care appointment, (ii) identify a next-available appointment slot in a plurality of doctor schedules stored on the at least one data storage device, (iii) book the next-available appointment slot as the urgent care appointment for the first patient, and (iv) provide a set of details of the next-available appointment to the patient electronic communication device.

In some examples, the set of details of the next-available appointment are a date and a time of day of the next-available appointment.

The set of details may include an indication that the patient will receive a phone call at the date and time of day from the first doctor.

The at least one processor may be further configured, following (iii), to notify, via the navigator style interface on a doctor electronic communications device, a doctor associated with the next-available appointment slot that the next-available appointment slot has been booked.

According to some aspects, there is provided a booking system, comprising (a) at least one processor; and (b) at least one data storage device communicatively coupled to the at least one processor, and (c) wherein the at least one processor is operable to: (i) generate a navigator style interface on a doctor electronic communication device of a first doctor, (ii) prompt, via the navigator style interface on the doctor electronic communication device, the first doctor to provide a first credential, (iii) prompt, via the navigator style interface on the doctor electronic communications device, the first doctor to provide a first doctor schedule, the first doctor schedule including at least one available appointment slot, (iv) receive the first credential and the first doctor schedule from the doctor electronic communication device, (v) incorporate the first credential and the first doctor schedule into a first doctor profile stored on the at least one data storage device, (vi) generate the navigator style interface on an administrator electronic communication device of an administrator user, (vii) prompt, via the navigator style interface on the administrator electronic communication device, the administrator user to review the first credential, (viii) receive an approval of the first doctor profile from the administrator electronic communication device, (ix) mark the first doctor profile as an approved profile on the at least one data storage device, (x) generate the navigator style interface on a patient electronic communication device of a first patient, (xi) present, via the navigator style interface on the patient electronic communication device, the first patient with a first booking option to book an urgent care appointment, (xii) receive, following (xi), a first option response from the patient electronic communication device requesting the urgent care appointment, (xiii) identify, following (ix), a next-available appointment slot in the first doctor schedule on the at least one data storage device, (xiv) book the next-available appointment slot for the first patient in the first doctor schedule on the at least one data storage device, and (xv) present, via the navigator style interface on the patient electronic communication device, the first patient with a set of details of the next-available appointment.

In some examples, the at least one processor is further operable to prompt, via the navigator style interface on the doctor electric communications device, the first doctor to provide a first doctor government-issued identification, and wherein the approval from the administrator electronic communications device is received after prompting, via the navigator style interface on the administrator electronic communication device, the administrator user to review the first doctor government-issued identification.

The first doctor government-issued identification may include a first image of a front side of an identification card and a second image of a rear side of the identification card.

The first credential may include a document issued by a third party doctor regulator.

The first credential may include at least one document issued by each of at least two third party doctor regulators.

The set of details of the next-available appointment may include a date and a time of day of the next-available appointment.

The set of details may include an indication that the patient will receive a phone call at the date and time of day from the first doctor.

The at least one processor may be further configured, following (xiv), to notify, via the navigator style interface on the doctor electronic communications device, the first doctor that the first doctor schedule on the at least one data storage device, has been updated.

The at least one processor may be further configured, prior to (xi), to: prompt, via the navigator style interface on the patient electronic communication device, the first patient to provide a first patient payment identifier; receive the first patient payment identifier from the first patient electronic communication device; and incorporate the first patient payment identifier into a first patient profile stored on the at least one data storage device.

The first patient payment identifier may be a third party identifier of the first patient issued by a third party payee or a set of personal payment information of the first patient.

The at least one processor may be operable to perform (xv) within one second of (xii).

The set of details may be presented via the navigator style interface on the patient electronic communication device as human-readable text.

According to some aspects, there is provided a booking system, comprising: (a) at least one processor; and (b) at least one data storage device communicatively coupled to the at least one processor, wherein the at least one data storage device stores at least one patient profile associated with at least one patient and at least one schedule associated with at least one doctor, and (c) wherein the at least one processor is operable to: (i) generate a navigator style interface on a patient electronic communication device of a first patient of the at least one patient, (ii) present, via the navigator style interface on the patient electronic communication device, the first patient with a first booking option to book an urgent care appointment, (iii) receive, following (ii), a first option response from the patient electronic communication device requesting the urgent care appointment, (iv) identify a next-available appointment slot in the at least one schedule on the data storage device, wherein the next-available appointment slot is in a first schedule of the at least one schedule, wherein the first schedule is associated with a first doctor of the at least one doctor, (v) book the next-available appointment slot for the first patient, (vi) identify a first patient profile of the at least one patient profile on the at least one data storage device, the first patient profile associated with the first patient, (vii) send, send following (vi), the first patient profile to a first doctor electronic communication device of the first doctor, and (viii) present, via the navigator style interface on the patient electronic communication device, the first patient with a set of details of the next-available appointment.

In some examples, the at least one processor is further operable to: (I) present, via the navigator style interface on the patient electronic communication device, the patient with a second booking option to book a routine appointment; (II) receive, following (I), a second option response from the patient electronic communication device requesting the routine appointment; (III) present, via the navigator style interface on the patient electronic communication device, the patient with a plurality of available appointment slots in the at least one schedule on the at least one data storage device; (IV) receive, following (III), a slot selection from the patient electronic communication device selecting a chosen available appointment slot of the plurality of available appointment slots on the at least one data storage device, wherein the chosen available appointment slot is in a second schedule of the at least one schedule, wherein the second schedule is associated with a second doctor of the at least one doctor; (V) book the second available appointment slot for the first patient; and (VI) send, following (V), the first patient profile to a second doctor electronic communication device of the second doctor.

The at least one processor may be operable to perform (V) within one second of (IV).

The set of details may be presented via the navigator style interface on the patient electronic communication device as human-readable text.

According to some aspects, there is provided a booking system, comprising (a) at least one processor; and (b) at least one data storage device communicatively coupled to the at least one processor, wherein the at least one data storage device stores at least one patient profile associated with at least one patient and at least one specialist doctor profile associated with at least one specialist doctor, and (c) wherein the at least one processor is operable to (i) generate a navigator style interface on a family doctor electronic communication device of a family doctor, (ii) present, via the navigator style interface on the family doctor electronic communication device, the family doctor with a specialist referral option to generate a referral of a first patient of the plurality of patients to the at least one specialist doctor, (iii) receive, following (ii), a referral response from the family doctor electronic communication device requesting the referral, (iv) present, via the navigator style interface on the family doctor electronic communication device, the family doctor with the at least one specialist doctor profile, (v) receive, following (iii), a selection from the family doctor electronic communication device of a first specialist doctor of the at least one specialist doctor, (vi) identify a first patient profile of the at least one patient profile, the first patient profile associated with the first patient, and (vii) send, following (v), the first patient profile to a first specialist doctor electronic communication device of the first specialist doctor.

In some examples, the at least one processor is further configured to generate a referral notice and present, via the navigator style interface on a first patient electronic communication device of the first patient, the referral notice to the first patient, wherein the referral notice includes information about the first specialist doctor.

The at least one processor may be further configured to: (I) present, via the navigator style interface on the first patient electronic communication device, the first patient with a booking option to book an appointment with the specialist doctor; (II) receive, following (I), a response from the first patient electronic communication device requesting the appointment with the specialist doctor; (III) present, via the navigator style interface on the first patient electronic communication device, the first patient with a plurality of available appointment slots in a first specialist doctor schedule of the first specialist doctor; (IV) receive, following (III), a slot selection from the first patient electronic communication device selecting an available appointment slot in the first specialist doctor schedule; (V) book the available appointment slot for the first patient; and (VI) present, via the navigator style interface on the first patient electronic communications device, the first patient with a confirmation of the booking.

The at least one processor may be operable to perform (VI) within one second of (IV) and the confirmation is presented via the navigator style interface on the patient electronic communication device as human-readable text.

### BRIEF DESCRIPTION OF THE DRAWINGS

The drawings included herewith are for illustrating various examples of systems, methods, and apparatus of the present specification. In the drawings:

FIG. 1 is a schematic diagram of a system for appointment booking;

FIG. 2 is a flow chart of a method of updating a health care professional profile;

FIG. 3 is a flow chart of a method of updating a patient profile;

FIG. 4 is a flow chart of a method of booking an urgent appointment;

FIG. 5 is a flow chart of a method of booking a routine appointment;

FIG. 6 is a flow chart of a referral method;

FIG. 7 is a first page of a navigator style interface displaying a first set of information;

FIG. 8 is a second page of the navigator style interface of FIG. 7;

FIG. 9 is a notice of the navigator style interface of FIG. 7;

FIG. 10 is the first page of the navigator style interface of FIG. 7 displaying a second set of information;

FIG. 11 is a patient calendar page of the navigator style interface of FIG. 7;

FIG. 12 is a patient appointment slot choice page of the navigator style interface of FIG. 7;

FIG. 13 is an extra-details page of the navigator style interface of FIG. 7;

FIG. 14 is a patient menu page of the navigator style interface of FIG. 7;

FIG. 15 is a notes page of the navigator style interface of FIG. 7 displaying a first set of information;

FIG. 16 is a new note page of the navigator style interface of FIG. 7;

FIG. 17 is the notes page of the navigator style interface of FIG. 7 displaying a second set of information;

FIG. 18 is an appointments page of the navigator style interface of FIG. 7;

FIG. 19 is a first portion of a details page of the navigator style interface of FIG. 7;

FIG. 20 is a second portion of the details page of the navigator style interface of FIG. 7;

FIG. 21 is a terms and conditions page of the navigator style interface of FIG. 7;

FIG. 22 is a patient settings page of the navigator style interface of FIG. 7;

FIG. 23 is a health care professional calendar page of the navigator style interface of FIG. 7;

FIG. 24 is a health care professional appointments page of the navigator style interface of FIG. 7;

FIG. 25 is a patient list page of the navigator style interface of FIG. 7;

FIG. 26 is a health care professional settings page of the navigator style interface of FIG. 7;

FIG. 27 is a health care professional menu page of the navigator style interface of FIG. 7;

FIG. 28 is a schedule page of the navigator style interface of FIG. 7;

FIG. 29 is an appointment set up page of the navigator style interface of FIG. 7;

FIG. 30 is a support and Frequently Asked Questions page of the navigator style interface of FIG. 7;

FIG. 31 is an administrator home page of the navigator style interface of FIG. 7;

FIG. 32 is the first page of the navigator style interface of FIG. 7 displaying a third set of information;

FIG. 33 is an identifier-entry page of the navigator style interface of FIG. 7;

FIG. 34 is a search page of the navigator style interface of FIG. 7;

FIG. 35 is a patient's summary page of the navigator style interface of FIG. 7;

FIG. 36 is a sign up page of the navigator style interface of FIG. 7;

FIG. 37 is an options page of the navigator style interface of FIG. 7;

FIG. 38 is a referral selection page of the navigator style interface of FIG. 7;

FIG. 39 is another details page of the navigator style interface of FIG. 7;

FIG. 40 is an acceptance page of the navigator style interface of FIG. 7; and,

FIG. 41 is a health care professional's patient summary page of the navigator style interface of FIG. 7.

### DETAILED DESCRIPTION

Various apparatus or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures, and components have not been described in detail so as not to obscure the embodiments generally described herein.

In some cases, the embodiments of the systems and methods described herein may be implemented in hardware or software, or a combination of both. In some cases, embodiments may be implemented in one or more computer programs executing on one or more programmable computing devices comprising at least one processor, a data storage device (including in some cases volatile and non-volatile memory and/or data storage elements), at least one input device, and at least one output device.

In some embodiments, each program may be implemented in a high-level procedural or object oriented programming and/or scripting language to communicate with a computer system. However, the programs can be implemented in assembly or machine language, if desired. In any case, the language may be a compiled or interpreted language.

In some embodiments, the systems and methods as described herein may also be implemented as a non-transitory computer-readable storage medium configured with a computer program, wherein the storage medium so configured causes a computer to operate in a specific and predefined manner to perform at least some of the functions as described herein.

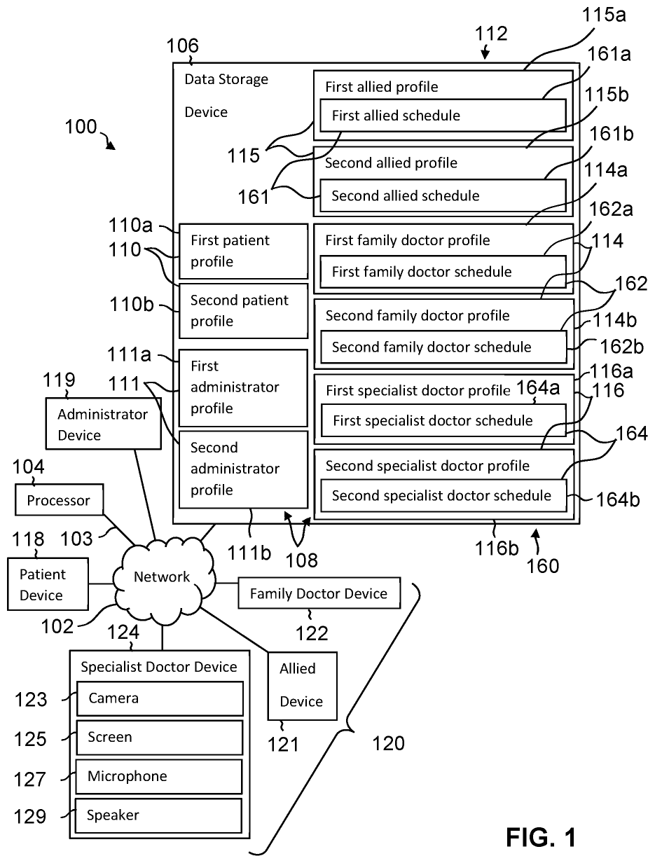

Referring to FIG. 1, illustrated therein is a system 100 for appointment booking. The system 100 is operable to be used to book appointments with health care professionals, as will be described in more detail elsewhere herein. In some examples, some or all information may be stored in a central database hosted on one or more remote servers (e.g., to assist with regulatory compliance and/or limit the usage of space on the mobile devices of users).

Components of the system 100 are connected via a network 102. The network 102 is an electronic communications network, and may be a wired and/or wireless electronic communications network. For example, the network 102 may be the Internet. The system 100 also includes at least one processor 104 and at least one data storage device 106 communicatively coupled to the processor 104 (e.g., via the network 102). For example, the system 100 may include a plurality of servers each including a processor 104 and/or a data storage device 106.

The data storage device 106 may store a plurality of user profiles 108. The plurality of user profiles 108 includes at least one patient profile 110 of at least one patient (e.g., a first patient profile 110a of a first patient and a second patient profile 110b of a second patient). Each of the patient profiles 110 includes a basic patient identifier of a patient (e.g., a name) and patient contact information (e.g., an email address or phone number). In some examples, each of the patient profile 110 includes a payment identifier, such as third party identifier of the first patient issued by a third party payee (e.g., an Ontario Health Insurance Plan (OHIP) identification number or other health insurance information) or a set of personal payment information of the patient (e.g., personal credit card details). The third party identifier may be issued by the third party and may identify the associated patent as an individual whose costs are covered, at least in part, by the third party. Each of the patient profiles 110 may also include at least one government issued identification (e.g., an identification number or a first image of a front side of an identification card such as a driver's license, and a second image of a rear side of the identification card). In some examples, a patient profile may be marked approved if it has been reviewed and allowed by an administrator (e.g., marked as allowed by an administrator device 119, discussed below), and may be marked pending if not yet approved.

The plurality of user profiles 108 also includes at least one administrator profile 111 of at least one administrator (e.g., a first administrator profile 111a of a first administrator and a second administrator profile 111b of a second administrator). Each of the administrator profiles 111 includes an administrator basic identifier (e.g., a name).

The plurality of user profiles 108 also includes at least one health care professional profile 112 of at least one health care professional (e.g., a doctor or allied health professional). Each of the at least one health care professional profile 112 includes a health care professional basic identifier (e.g., a name) and health care professional contact information (e.g., an email address or phone number). In some examples, each of the health care professional profiles 112 includes a credential issued by a third party and identifying the associated health care professional as an individual associated with the third party. The credential may be, for example, an insurance credential (e.g., insured by the third party to practice medicine) or a basic licensing credential (e.g., a College of Physicians and Surgeons of Ontario (CPSO) identification number). The credential may include one or more documents issued by the third party (e.g., a document issued by the Canadian Medical Protective Association (CMPA)). Each of the health care professional profiles 112 may also include at least one government issued identification (e.g., a first image of a front side of an identification card such as a driver's license, and a second image of a rear side of the identification card). In some examples, a health care professional profile may be marked approved if it has been reviewed and allowed by an administrator (e.g., marked as allowed by an administrator device 119, discussed below), and may be marked pending if not yet approved.

The health care professional profiles 112 may include at least one family doctor profile 114 (e.g., a first family doctor profile 114a and a second family doctor profile 114b), at least one allied health care professional profile 115 (e.g., a first allied health care professional profile 115a and a second allied health care professional profile 115b), and at least one specialist doctor profile 116 (e.g., a first specialist doctor profile 116a and a second specialist doctor profile 116b). Each of the specialist doctor profiles 116 includes a third party advanced license credential (e.g., certified by the Royal College of Physicians and Surgeons of Canada in a specialty or subspecialty such as gynecology, cardiology, orthopedic surgery, or dermatology) in addition to the third party basic licensing credential. Each of the family doctor profiles 114 includes the third party basic licensing credential without a further third party advanced license credential. In some examples, each of the family doctor and specialist doctor profiles includes a third party basic licensing credential that includes a license to practice medicine as a doctor, while each allied health care professional profile includes a third party basic licensing credential that does not include a license to practice medicine as a doctor.

The system 100 also includes at least one patient device 118, at least one administrator device 119, and at least one health care professional device 120. The at least one health care professional device 120 may include at least one family doctor device 122, at least one allied health care professional device 121, and at least one specialist doctor device 124. The patient device 118 is a device (e.g., a mobile device such as a smartphone) associated with a patient profile 110 (e.g., signed into the patient profile 110). Similarly, the administrator device 119 is associated with an administrator profile 111, and the health care professional device 120 is a device associated with a health care professional profile 112. The family doctor device 122 is a device associated with a family doctor

profile 114, the allied health care professional device 121 is a device associated with an allied health care professional profile 115, and the specialist doctor device 124 is a device associated with a specialist doctor profile 116.

Each of the devices 118, 119, 122, 121, 124 is an electronic communications device. One or more of the device 118, 119, 122, 121, 124 may be a mobile device, such as a smartphone or tablet. One or more of the devices 118, 119, 122, 121, 124 may be a desktop device, such as a desktop computer. In some examples, the patient device is a mobile device (e.g., a smartphone) accessing the system through navigator style interface, the navigator style interface includes a mobile application. The administrator device 119, family doctor device 122, allied health care professional device 121, and/or specialist doctor device 124 may also be a mobile device accessing the system through a mobile application. For example, the patient device 118 may be a smartphone accessing the system 100 through a mobile application and each of the each of the family doctor device 122, allied health care professional device 121, and the specialist doctor device 124 may also be a smartphone accessing the system 100 through a mobile application. However, in some examples, the patient device 118 is a smartphone accessing the system 100 through a mobile application and each of the family doctor device 122, allied health care professional device 121, and the specialist doctor device 124 is a desktop computer.

The processor 104, data storage device 106, patient device 118, administrator device 119, family doctor device 122, allied health care professional device 121, and specialist doctor device 124 are interconnected through the network 102. Each of these devices is connected to the network 102 via a connection 103, which may be wired or wireless (e.g., a Wi-Fi™ connection, a Bluetooth™ connection, or a cellular connection).

In some examples, the processor 104 includes one or more processors of the patient device 118, one or more processors of the administrator device 119, one or more processors of the family doctor device 122, one or more processors of the allied health care professional device 121, and/or one or more processors of the specialist doctor device 124. In some examples, the data storage device 106 includes one or more data storage devices of the patient device 118, one or more data storage devices of the administrator device 119, one or more data storage devices of the family doctor device 122, one or more data storage devices of the allied health care professional device 121, and/or one or more data storage devices of the specialist doctor device 124.

In some examples, the patient device 118, the administrator device 119, the family doctor device 122, the allied health care professional device 121, and/or the specialist doctor device 124 includes a camera 123, a screen 125, a microphone 127, and/or a speaker 129. In some examples, each of the devices 118, 119, 122, 121, 124 includes at least the screen 125 to present a navigator style interface, as discussed further elsewhere herein, and the screen 125 is a touchscreen.

The device 118, 119, 122, 121, 124 with the camera 123, the screen 125, the microphone 127, and/or the speaker 129 may be operable to make and/or receive audio and/or video calls with another device 118, 119, 122, 121, 124. The system 100 may be used also to conduct an appointment after it has been booked (e.g., via video or audio call between the patient device 118 and the health care professional device 120).

The processor 104 is operable to generate the navigator style interface on the devices 118, 119, 122, 121, 124 to collect from and/or provide information to the users. For example, the processor 104 may use the navigator style interface to generate and/or update one or more of the profiles 108.

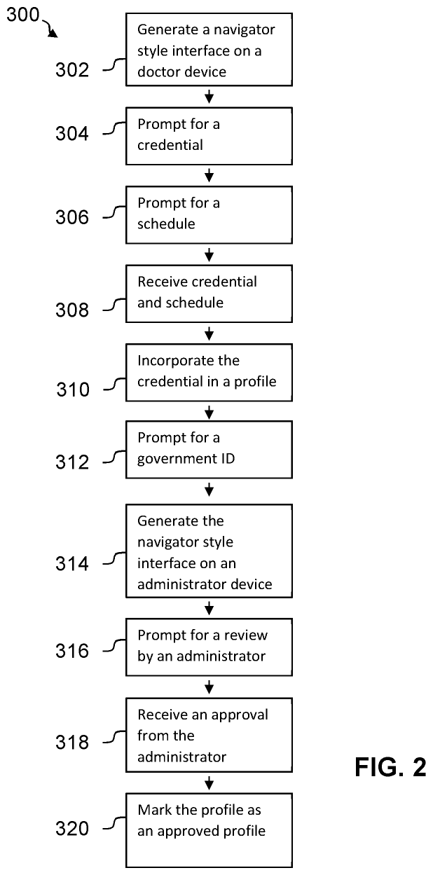

Referring to FIG. 2, illustrated is a method 300 of updating a health care professional profile 112. The health care professional profile 112 may be initially generated with log in information, and may include basic information such as contact information and identification information (e.g., an email address and a name).

The processor 104 is operable to, at step 302, generate the navigator style interface on a health care professional electronic communication device 120 of a first health care professional. Examples of pages of the navigator style interface are discussed further elsewhere herein. The processor 104 is operable to, at step 304, prompt, via the navigator style interface on the health care professional electronic communication device, the first health care professional to provide a first credential. For example, the first health care professional may be presented with an upload page requiring that a credential document from a third party licensing authority be uploaded before the first health care professional can make themselves available for appointments. The processor 104 is operable to, at step 306, prompt, via the navigator style interface on the health care professional electronic communications device, the first health care professional to provide a first health care professional schedule, the first health care professional schedule including at least one available appointment slot. For example, the first health care professional may be presented with an appointments screen in which they can enter the details of one or more appointment slots during which they can meet with a patient (e.g., phone a patient from the health care professional device 120 and/or a different device to conduct a virtual appointment).

The processor 104 is operable to, at step 308, receive the first credential and the first health care professional schedule from the health care professional electronic communication device 120. The processor 104 is operable to, at step 310, incorporate the first credential and the first health care professional schedule into a first health care professional profile 112 (e.g., the first family doctor profile 114a) stored on the data storage device 106.

The processor 104 may be operable to, at step 312, prompt, via the navigator style interface on the health care professional electric communications device 120, the first health care professional to provide a first health care professional government-issued identification. For example, the first health care professional government-issued identification may include a first image of a front side of an identification card and a second image of a rear side of the identification card (e.g., a driver's license). The processor 104 may also be operable to receive the government-issued identification and incorporate the government-issued identification into the first health care professional profile 112 stored on the data storage device 106.

The processor 104 is operable to, at step 314, generate the navigator style interface on an administrator electronic communication device 119 of an administrator user. The processor 104 is operable to, at step 316, prompt, via the navigator style interface on the administrator electronic communication device 119, the administrator user to review the first credential. The processor 104 is operable to, at step 318, receive an approval of the first health care professional profile from the administrator electronic communication device 119. The approval from the administrator electronic communications device 119 may be received after prompting, via the navigator style interface on the administrator electronic communication device 119, the administrator user to review the first health care professional government-issued identification as well. The processor 104 is operable to, at step 320, mark the first health care professional profile 112 as an approved health care professional profile 112 on the data storage device 106. In some examples, patients are only matched to appointment slots that are associated with approved health care professional profiles 112.

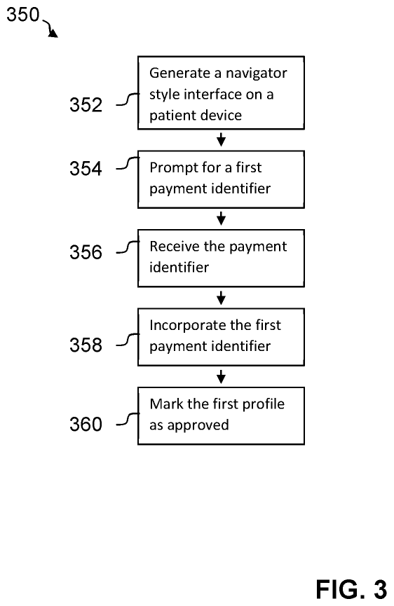

Referring to FIG. 3, illustrated is a method 300 of updating a patient profile 110. The patient profile 110 may be initially generated with log in information, and may include basic information such as contact information and identification information (e.g., an email address and a name).

The processor 104 is operable to, at step 352, generate the navigator style interface on a patient electronic communication device 118 of a first patient. The processor 104 is operable to, at step 354, prompt, via the navigator style interface on the patient electronic communication device 118, the first patient to provide a first patient payment identifier. For example, the patient may be asked for a third party identifier (e.g., an OHIP identifier) or a set of personal payment information (e.g., credit card information), in some examples the patient may be asked for both so that the set of personal payment information can be used for any services not covered by the third party. The processor 104 is operable to, at step 356, receive the first patient payment identifier from the first patient electronic communication device 118. The processor 104 is operable to, at step 358, incorporate the first patient payment identifier into a first patient profile 110 stored on the data storage device 106.

The processor 104 is operable to, at step 360, verify that the payment identifier is valid and mark the first patient profile 110 as an approved patient profile 110. In some examples, only approved patient profiles 110 and associated devices 118 may be used to book appointments.

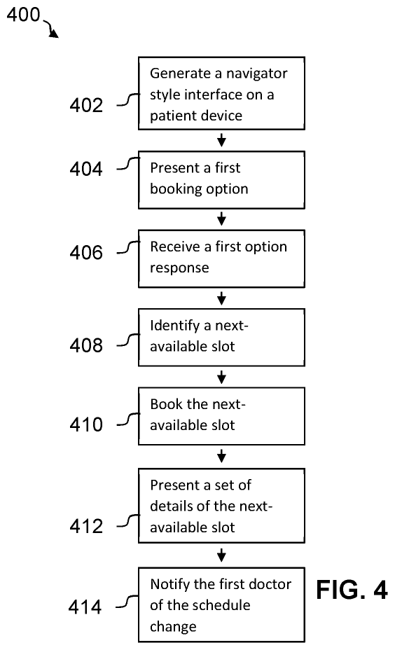

Referring to FIG. 4, illustrated is a method 400 of booking an appointment. Method 400 allows a patient to set up an urgent appointment. The processor 104 is operable to, at step 402, generate the navigator style interface on a patient electronic communication device 118 of a first patient. The processor 104 is operable to, at step 404, present, via the navigator style interface on the patient electronic communication device 118, the first patient with a first booking option to book an urgent care appointment. For example, the patient may be offered an urgent appointment for non-emergency needs that the patient would still like to have addressed as soon as possible.

The processor 104 is operable to, at step 406, receive, following step 404, a first option response from the patient electronic communication device 118 requesting the urgent care appointment. The processor 104 is operable to, at step 408, identify, following step 320, a next-available appointment slot in the first health care professional schedule (e.g., reviewing each of the family doctor schedules 162 and finding that the next-available appointment is a slot in the second family doctor schedule 162b) on the data storage device 106. The search may be limited to the schedules of family doctors (e.g., if allied health care professionals and specialists are available via other scheduling options, such as by searching for allied health care professional or being referred to specialists). In some examples, this may be limited to the next-available slot within a predetermined window (e.g., at least 15 minutes from the present booking time but not more than two hours), as discussed further below. In some examples, where more than one doctor is availed at a particular time, the processor 104 may be operable to randomly assign one of the doctors. Randomly assigning a doctor rather than allowing a patient to choose may reduce personal connections between patients and doctors. However, in some examples, the patient can limit the processor 104 to searching for appointments associated with one or more patient-selected doctors (e.g., a doctor associated with the patient's profile as the patient's family doctor and/or a doctor matching one or more criteria provided by the patient such as location criteria). In some examples, limiting the search to appointments associated with one or more patient-selected doctors is only an option for non-urgent appointments.

The processor 104 is operable to, at step 410, book the next-available appointment slot for the first patient in the first health care professional schedule (e.g., the second family doctor schedule 162b, as noted above) on the data storage device 106. The processor 104 is operable to, at step 412, present, via the navigator style interface on the patient electronic communication device 118, the first patient with a set of details of the next-available appointment. The set of details may be a date and a time of day, and may include an indication that the patient will receive a phone call or video call at the data and time of day from the first health care professional to conduct the appointment over the phone or by video call.

In some examples, the system 100 and/or processor 104 is operable to perform step 412 within a predetermined time spread of step 406, such as within 10 seconds, within 5 seconds, within 1 second, or within 0.1 seconds. Rather than wait several seconds or minutes for a receptionist to review possible times and dates, which may be particularly troubling in an urgent situation, the system 100 may generate the booking rapidly.

In some examples, the set of details are presented via the navigator style interface on the patient electronic communication device as human-readable text. Rather than hearing the details spoken by a receptionist, which may be difficult to remember, understand, or take note of, the system 100 may generate a textual notice that the user can view. In some examples, the information is saved and can be reviewed again later on.

The processor 104 may also be operable to, at step 414, notify, via the navigator style interface on the health care professional electronic communications device 120, the first health care professional associated with the first health care professional schedule that the first health care professional schedule on the data storage device 106 has been updated. In some examples, the processor 104 is further operable to connect the health care professional device 120 associated with the first health care professional schedule with the patient device 118 associated with the first patient (e.g., by audio or video call) for the appointment to be conducted. For example, the processor 104 may retrieve contact information (e.g., a phone number or information needed for a video call via a third party platform such as Facebook™) from the patient profile and use this to initiate a connection (e.g., an audio and/or video call) between the health care professional's device and the patient's device (e.g., in repose to an initiation signal such as a confirmation from the health care professional that they are ready to proceed and/or an indication from a system clock indicating the system time matches the appointment time). However, in some examples the health care professional is provided with the contact information and initiates the call manually (e.g., is provided with a phone number and then enters that into the health care professional's device or another device to initiate the call).

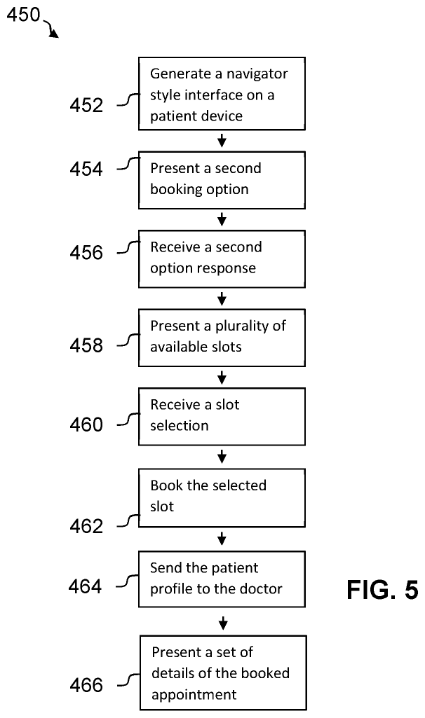

Referring to FIG. 5, illustrated is a method 450 of booking an appointment. Method 450 allows a patient to set up a routine appointment rather than, or in addition to, allowing the patient to set up an urgent appointment. The processor 104 is operable to, at step 452, generate the navigator style interface on the patient device 118. The processor 104 is operable to, at step 454, present, via the navigator style interface on the patient electronic communication device 118, the patient with a second booking option to book a routine appointment. In some examples, the patient is presented with both the option to book a routine appointment and the option to book an urgent appointment as discussed above.

The processor 104 is operable to, at step 456, receive, following step 454, a second option response from the patient electronic communication device 118 requesting the routine appointment. The processor 104 is operable to, at step 458, present, via the navigator style interface on the patient electronic communication device 118, the patient with a plurality of available appointment slots in the at least one schedule (e.g., allowing the patient to see all available slots in the family doctor schedules 162) on the data storage device 106. This may be shown to the patient in the form of a calendar of days from which the patient may select a day and then see available slots on that day, as discussed further elsewhere herein.

The processor 104 is operable to, at step 460, receive, following step 458, a slot selection from the patient electronic communication device 118 selecting a chosen available appointment slot of the plurality of available appointment slots on the data storage device 106, wherein the chosen available appointment slot is in a second schedule of the at least one schedule (e.g., a slot in the first family doctor schedule 162a), wherein the second schedule is associated with a health care professional of the at least one health care professional. In some examples, where more than one health care professional is availed at a particular time, the processor 104 may be operable to randomly assign one of the health care professionals. Randomly assigning a health care professional rather than allowing a patient to choose may reduce personal connections between patients and health care professionals. However, in some examples, the patient can limit the processor 104 to searching for appointments associated with one or more patient-selected health care professionals (e.g., a health care professional associated with the patient's profile as the patient's family doctor and/or a health care professional matching one or more criteria provided by the patient such as location criteria). The processor 104 is operable to, at step 462, book the second available appointment slot for the first patient. The processor 104 may be operable to perform step 462 within the predetermined time spread of step 460 discussed above.

The processor 104 is operable to, at step 464, send, following step 462, the first patient profile to a health care professional electronic communication device 120 of the health care professional. For example, the patient profile may be and/or include a phone number, and the phone number may be sent to the device of the health care professional so that the health care professional may phone the patient to conduct the appointment. The processor 104 may be operable to, at step 466, present, via the navigator style interface on the patient device 118, the patient with a set of details of the booked appointment. The set of details may be presented via the navigator style interface on the patient electronic communication device 118 as human-readable text.

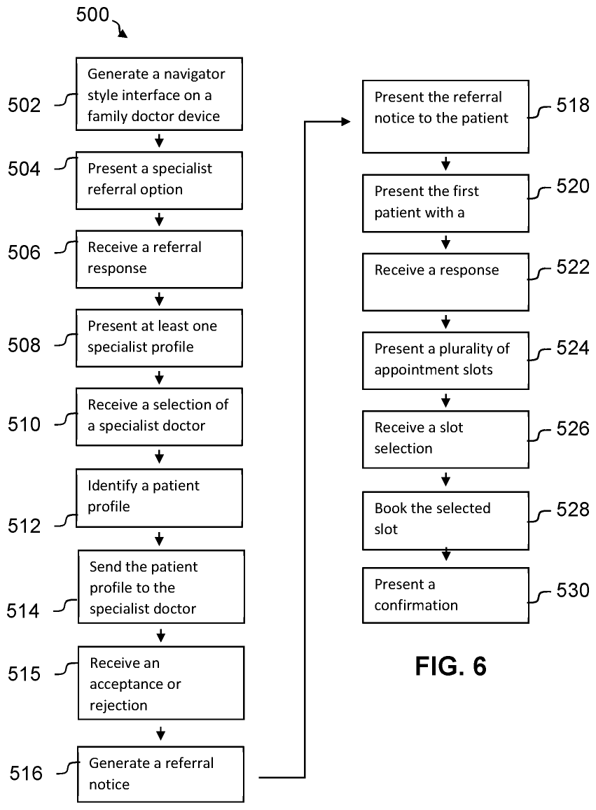

Referring now to FIG. 6, illustrated is a referral method 500. Method 500 may be used by a family doctor to refer a patient to a specialist doctor and/or an allied health care professional. In some examples, a patient can only schedule an appointment with a specialist doctor once a family doctor has made a referral. The processor 104 is operable to, at step 502, generate a navigator style interface on a family doctor electronic communication device 122 of a family doctor. The processor 104 is operable to, at step 504, present, via the navigator style interface on the family doctor electronic communication device 122, the family doctor with a referral option to generate a referral of a first patient of the plurality of patients to the at least one specialist doctor and/or allied health care professional.

In some examples, step 504 is associated with an appointment. For example, the specialist referral option may be presented to a family doctor when the family doctor finishes an appointment and/or marks an appointment as complete (e.g., an option and/or soft button to “mark complete” and a separate option and/or soft button to “mark complete and refer to a specialist”). The referral option may also be available for past appointments. For example, the family doctor may access past appointments and open the details of a cancelled or completed appointment and have the option to refer to a specialist or an allied professional (e.g., a soft button to “refer to a specialist” or a soft button to “refer to an allied professional”).

The processor 104 is operable to, at step 506, receive, following step 504, a referral response from the family doctor electronic communication device 122 requesting the referral. The processor 104 is operable to, at step 508, present, via the navigator style interface on the family doctor electronic communication device 122, the family doctor with the specialist doctor profiles 116 and/or allied health care professional profiles 115.

The processor 104 is operable to, at step 510, receive, following step 506, a selection from the family doctor electronic communication device 122 of a first specialist doctor of the at least one specialist doctor (e.g., a selection of the second specialist doctor profile 116b) and/or a first allied health care professional. The family doctor may be required to fill in information for the specialist and/or allied health care professional to be included with the request, such as notes regarding the reason for the referral and/or information about the patient. The processor 104 may be operable to, at step 512, identify a first patient profile of the at least one patient profile (e.g., the second patient profile 110b), the first patient profile associated with the first patient. The processor 104 may be operable to, at step 514, send, following step 510, the first patient profile 110 to a first specialist doctor electronic communication device 124 of the first specialist doctor and/or the first allied health care professional device 121 of the first allied health care professional. In some examples, at step 514, the processor 104 sends the information filled in by the family doctor, with or without the information about the first patient from the patient's profile (e.g., the processor 104 may not first conduct step 512). At step 515, the processor 104 receives an acceptance or rejection of the request from the specialist and/or allied health care professional.

The processor 104 may be operable to, at step 516, generate a referral notice and, at step 518, present, via the navigator style interface on a first patient electronic communication device 118 of the first patient, the referral notice to the first patient. In some examples, step 516 may take place prior to step 515. The referral notice may include information about the first specialist doctor or simply a notice that a referral has been generated. In some examples, step 516 may take place after step 515 or may include a first notice sent prior to step 515 and a second notice sent after step 515. For example, the notice may be used to let the patient know that they are being referred to a specialist, and to let the patient know who the specialist is, what the referral is for, and/or how to contact the specialist. In another example, a first notice prior to step 515 may inform the patient that a referral has been made, and a second notice after step 515 may information the patent regarding who the specialist is.

The processor 104 may be operable to, at step 520, present, via the navigator style interface on the first patient electronic communication device 118, the first patient with a booking option to book an appointment with the specialist doctor and/or allied health care professional. The processor 104 may be operable to, at step 522, receive, following step 510, a response from the first patient electronic communication device 118 requesting the appointment with the specialist doctor and/or allied health care professional.

The processor 104 may be operable to, at step 524, present, via the navigator style interface on the first patient electronic communication device 118, the first patient with a plurality of available appointment slots in a first specialist doctor schedule 164 of the first specialist doctor and/or a first allied health care professional schedule 161 of the first allied health care professional. The processor 104 may be operable to, at step 526, receive, following step 524, a slot selection from the first patient electronic communication device 118 selecting an available appointment slot in the first specialist doctor schedule 164 and/or first allied health care professional schedule 161.

The processor 104 may be operable to, at step 528, book the available appointment slot for the first patient. The processor 104 may be operable to, at step 530, present, via the navigator style interface on the first patient electronic communications device, the first patient with a confirmation of the booking (e.g., including time and date information for the appointment). The confirmation may be saved so that the patient can review the confirmation later. In some examples, the processor 104 is operable to perform step 530 within the predetermined time spread of step 546 discussed above. In some examples, the confirmation is presented via the navigator style interface on the patient electronic communication device 118 as human-readable text.

The methods are described further below with reference to pages of the navigator style interface.

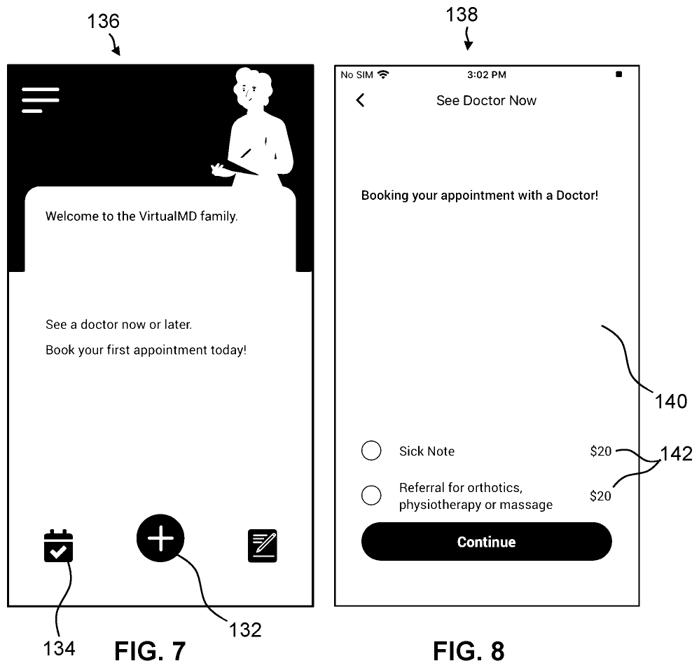

Referring now to FIG. 7, the processor 104 is operable to generate the navigator style interface on a first patient communication device 118 associated with the first patient profile 110a of the plurality of patient profiles, the first patient profile 110a being the profile of a first patient. The processor 104 is further operable to present on the first patient communication device 118, via the navigator style interface, a first booking option 132 to book an urgent care appointment. In some examples, the processor 104 is also operable to present on the first patient communication device 118, via the navigator style interface, the first patient with a second booking option 134 to book a routine appointment. The first and second booking options 132, 134 may be presented together on a first page 136 of the navigator style interface, as in the illustrated example.

If the first patient needs an urgent care appointment (e.g., they wish to book the next available appointment), they may choose the first booking option 132. If the patient selects the first booking option 132, the processor 104 receives a first option response from the first patient requesting the urgent care appointment.

The processor 104 is operable to communicate with the data storage device 106. The data storage device 106 may store the health care professional schedules 160. Each health care professional schedule 160 may be associated with a health care professional profile 112 and may contain one or more appointment slots (e.g., predetermined periods of time on a predetermined day, such as 9:00 am to 10:00 am on a given calendar day). Any given appointment slot may be booked for a particular individual or task or may be available (i.e., not yet booked).

In some examples, the at least one health care professional schedule 160 is a plurality of health care professional schedules 160, and may include at least one family doctor schedule 162 (e.g., a first family doctor schedule 162a associated with a first family doctor profile 114a and a second family doctor schedule 162b associated with a second family doctor profile 114b), at least one allied health care professional schedule 161 (e.g., a first allied health care professional schedule 161a associated with a first allied health care professional profile 115a and a second allied health care professional schedule 161b associated with a second allied health care professional profile 115b), and at least one specialist doctor schedule 164 (e.g., a first specialist doctor schedule 164a associated with a first specialist doctor profile 116a and a second specialist doctor schedule 164b associated with a second specialist doctor profile 116b).

The processor 104 is operable to identify a next-available appointment slot of the schedules 160. In some examples, the appointment slot is limited to a slot in a family doctor schedule 162. For example, the health care professional schedules 160 may include the first family doctor schedule 162a associated with the first family doctor profile 114a and the second family doctor schedule 162b associated with the second family doctor profile 114b. The processor 104 may review both the first and second family doctor schedules 162a, 162b, and determine that the available appointment slot closest in time to the time of the processing operation is a slot in the first family doctor schedule 162a. The processor 104 may limit the review to slots that are at least a first predetermined time limit in the future, e.g., at least 10 minutes in the future, at least 30 minutes in the future or at least an hour in the future. The processor 104 may then select that slot as the next-available appointment slot.

The processor 104 may be configured to search for available slots that are not more than a second predetermined time limit from the booking time, e.g., not more than a week, not more than 3 days, not more than 2 days, or not more than 24 hours. If the processor 104 is not able to find an available slot within the first and second predetermined time limits the processor 104 may be operable to generate an error notification and provide the error notification to the first patient via the navigator style interface on the first patient device 118. For example, the error notification may indicate that an urgent appointment is not available within a predetermined period of time (e.g., 5 days, 24 hours, 12 hours, etc.), and suggest that the patient book a routine appointment, as discussed further below.

Referring now to FIG. 8, in response to receiving the first option response, the processor 104 may present a second page 138 via the navigator style interface. The processor 104 may be operable to accept one or more notes or add-on services requests via the navigator style interface. For example, the processor 104 may be operable to accept, via the second page 138, a note entered into a note space 140 or information about a requested add-on service selected from an add-on service list 142. In some examples, the user may be charged for an add-on service, such as if the add-on service is not covered by a third party (e.g., a sick note not covered by OHIP). In some examples, costs of an appointment are split between the first patient and a third party.

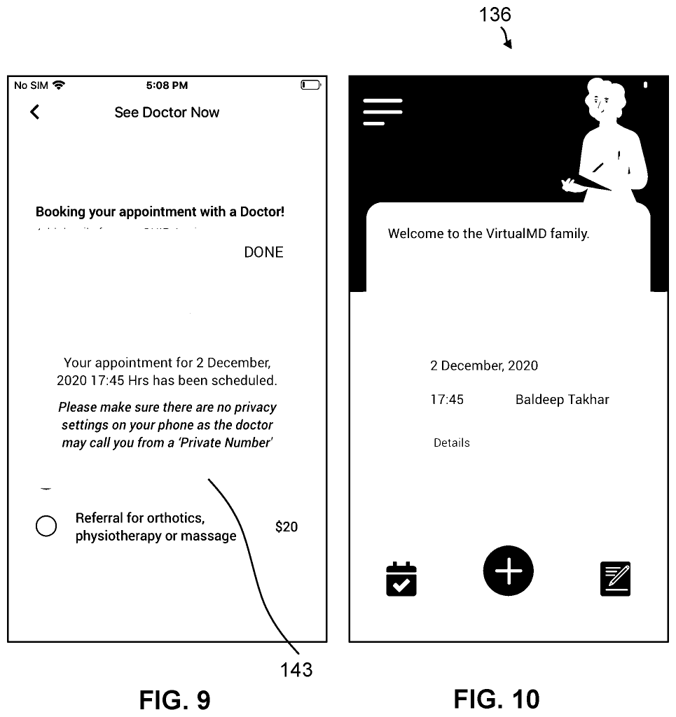

Referring to FIG. 9, the processor 104 is operable to book the next-available appointment slot for the patient and update the first health care professional schedule. The processor 104 is operable to send a notice 143 to the first patient communications device 108 to confirm that the next-available appointment slot is booked and to provide time and date information. In some examples, the confirmation or notice 143 is the first time the patient is provided with the time and date information when they have chosen to book an urgent appointment. In some examples, one or more item of information is provided later, such as in a phone call initiated by the health care professional or an email.

In some examples, the processor 104 is also operable to identify the first patient profile, identify a first health care professional profile associated with the first health care professional schedule, and send the first patient profile to a first health care professional communication device associated with the first health care professional profile. For example, the health care professional may receive a phone number and/or email address of the patient so the health care professional is able to reach out and provide further information such as location information. In some embodiments, the health care professional may be able to conduct the appointment through the navigator style interface (e.g., the processor 104 may be operable to receive a command from the health care professional to initiate a call to the patient, and the processor 104 may connect the call so that the appointment can be conducted).

Referring now to FIG. 10, once an appointment is booked, the first page 136 of the navigator style interface on the patient device 118 may be updated to show appointment details.

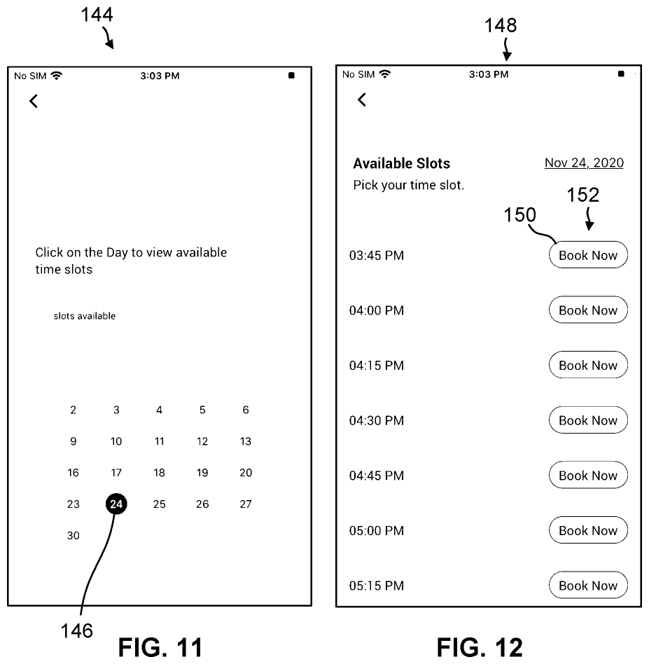

Referring now to FIGS. 11 and 12, if the first patient selects the second booking option 134, the processor 104 receives a second option response from the first patient. The second option response is a request for the routine appointment (i.e., not urgent). In response, the processor 104 is operable to present the patient with a plurality of available appointment slots in the health care professional schedules 160 via the navigator style interface. For example, the processor 104 may present the first patient with a patient calendar page 144 on the patient device 118, from which the first user may select a day 146 on which they would like to have the appointment. The processor 104 may then present an appointment slot choice page 148 via the navigator style interface on the patient device 118, from which the first patient may choose an available time slot to book from among a plurality of available appointment slots 152.

The processor 104 is operable to receive a slot selection 150 from the first patient. The slot selection 150 is a choice from among the plurality of available appointment slots 152, and is a slot in a health care professional schedule 160. Again, the patient may be limited to booking with a family doctor in some embodiments. For example, the slot selection 150 may be a slot in the second family doctor schedule 162b associated with the second family doctor profile 114b.

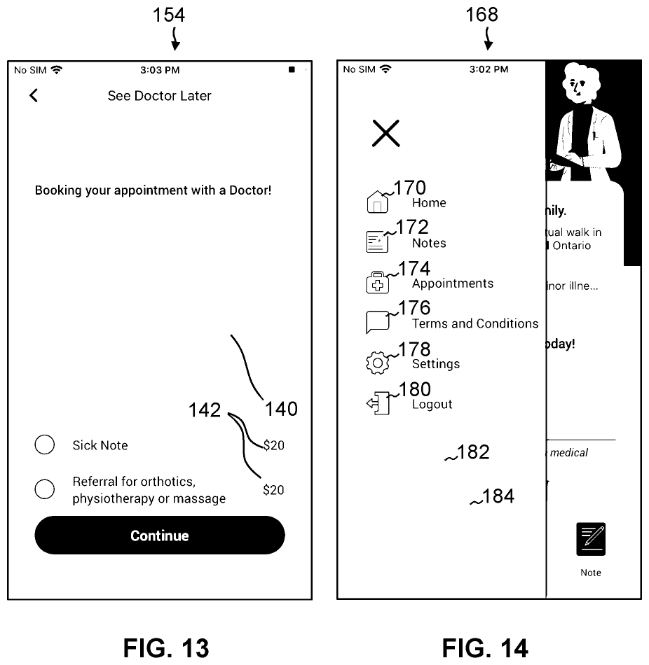

Referring to FIG. 13, the processor 104 may again be operable to accept one or more notes or add-on services requests via the navigator style interface, such as a note entered into a note space 140 or information about a requested add-on service selected from an add-on service list 142 of an extra-details page 154. The processor is operable to book the slot selection for the first patient and send the first patient profile of the first patient to the doctor communications device 122 associated with the doctor schedule of the slot selection.

Referring to FIG. 14, the processor 104 may also be operable to present a patient menu page 168 via the navigator style interface on the patient device 118. The patient may be able to select, via the patient menu page 168, a home option 170 to return to the first page 136, a notes option 172 to enter a personal note for their own reference, an appointments option 174 to view upcoming and/or past appointments, a terms and conditions option 176 to view terms and conditions, a settings option 178 to view settings, a logout option 180 to log out, a privacy policy option 182 to view a privacy policy, or a support and Frequently Asked Questions option 184 to view supporting information.

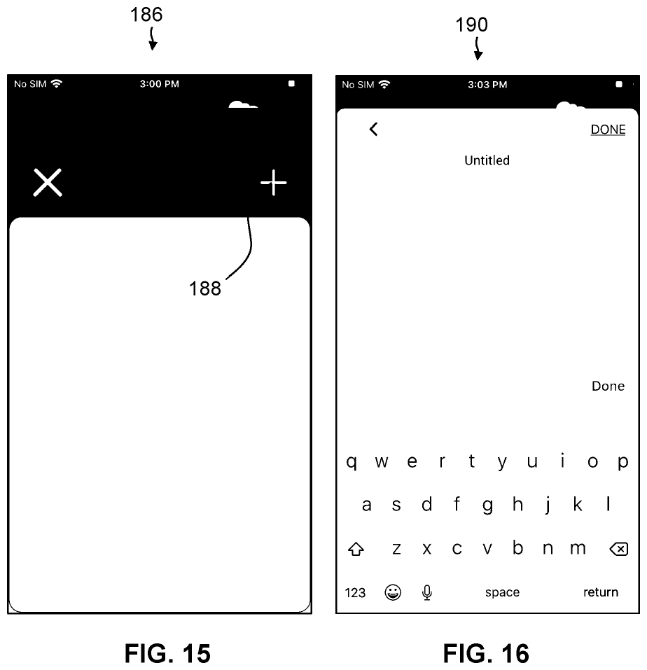

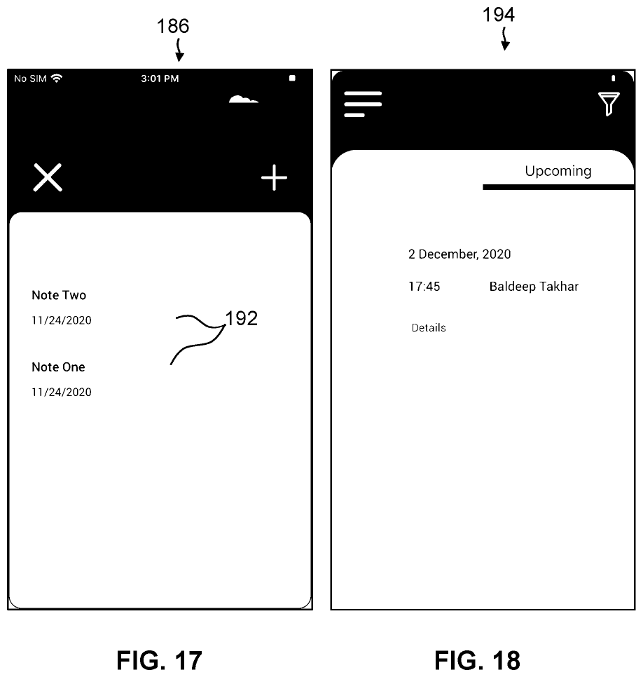

Referring now to FIGS. 15 to 17, if the patient selects the notes option 172 the processor 104 is operable to generate a notes page 186 on the patient device 118. The patient may be able to indicate a desire to add a note via the add note option 188, following which the processor 104 may provide a new note page 190 via which the user may enter a note. The notes page 186 may then be updated to included accessible notes 192. In some examples, notes entered by the patient are stored on a local data storage device on the patient device 118 rather than on the data storage device 106 of the system 100.

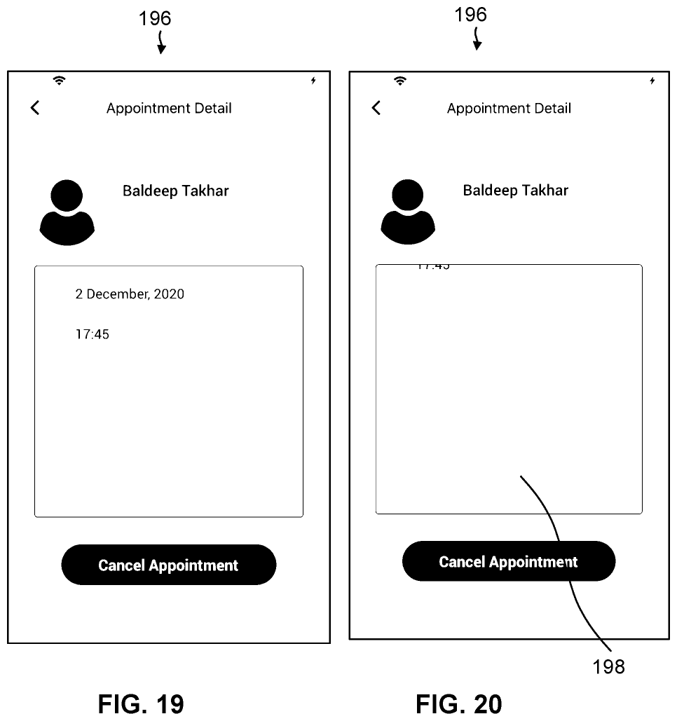

Referring now to FIGS. 18 to 20, if the patient selects the appointments option 174 the processor 104 is operable to generate a patient appointments page 194 on the patient device 118. The patient appointments page 194 may show the patient details of past and/or upcoming appointments. The patient may select a listed appointment, and in response, the processor 104 may be operable to present, via the navigator style interface on the patient device 118, details of the appointment. For example, a details page 196 may be presented, showing time and date details and any notes or other notifications 198. For example, the patient may again be reminded that the appointment may require certain settings on the patient device 118 so that the doctor can call to conduct the appointment by phone.

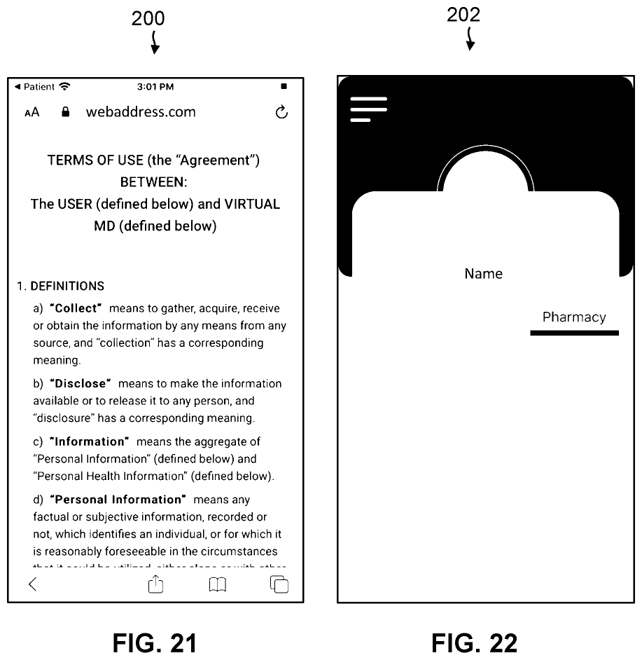

Referring now to FIG. 21, if the patient selects the terms and conditions option 176 the processor 104 is operable to generate a terms and conditions page 200 on the patient device 118. The terms and conditions page 200 may show the patient details of a current terms and conditions agreement that the patient must abide by.

Referring now to FIG. 22, if the patient selects the settings option 178 the processor 104 is operable to generate a patient settings page 202 on the patient device 118. The patient settings page 202 may show the patient details included in the patient's profile, such as basic details (e.g., name, birth date, third party payment identifier, and address), account details (e.g., password, payment information, and phone number), or pharmacy information (e.g., pharmacy name, pharmacy phone number, pharmacy address, or pharmacy fax number).

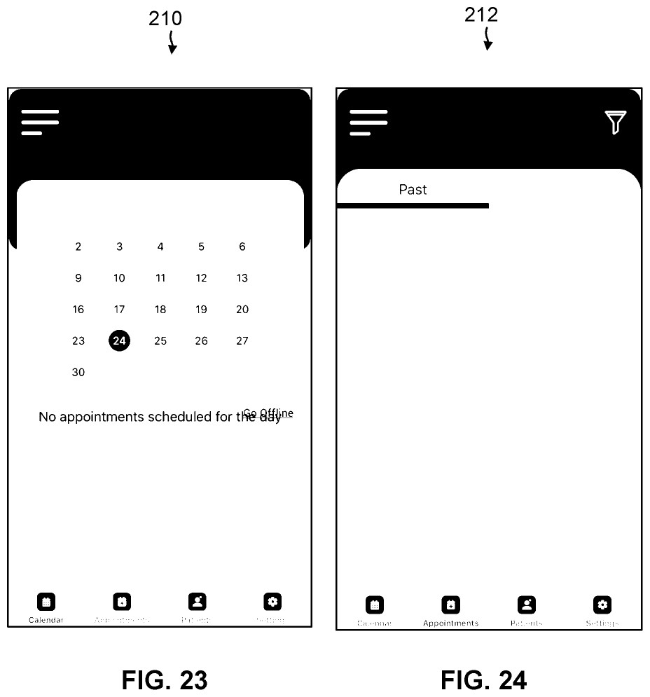

Referring to FIG. 23, the processor 104 is operable to generate the navigator style interface on a health care professional communication device 120 of a health care professional (e.g., a family doctor device 122 or a specialist doctor 124). The navigator style interface includes a health care professional calendar page 210. The health care professional calendar page 210 may show the health care professional a calendar, through which the health care professional can access appointment details and slots.

Referring to FIG. 24, the navigator style interface includes an appointments page 212. The health care professional appointments page 212 shows the health care professional a list of past and upcoming appointments. Again, an appointment in the list may be selected to provide further details about the appointment, similar to the patient appointments page 194. An appointment may have the status of ‘Pending’, ‘Completed’, or ‘Canceled’. In some examples, any appointment booked by a patient is Pending until the health care professional actively switches the status to Completed or Cancelled (e.g., navigates via the health care professional device 120 to a soft button associated with the appointment and clicks it to indicate that the appointment is complete). Where the system 100 is not used to conduct the appointments (e.g., the health care professional receives a phone number and timing details via system 100 but then calls the phone number without using system 100) it may be particularly important for the health care professional to update the system 100 to indicate whether or not the appointment was conducted, since the system 100 cannot be updated automatically. Simply determining an appointment to be complete because the time it was booked for has past may result in inaccurate updating, as the appointment may not have taken place (e.g., a call to the phone number available for the patient was not answered). A patient may also be able to mark an appointment as Cancelled (e.g., via patient device 118), e.g., if they no longer wish to have the appointment. In some examples, an appointment may only be marked Complete via the navigator style interface on a health care professional device 120 (i.e., not by a patient). In some examples, a health care professional device 120 may also be used to add notes to an appointment (e.g., to a completed appointment, which may be incorporated into a user profile 108).

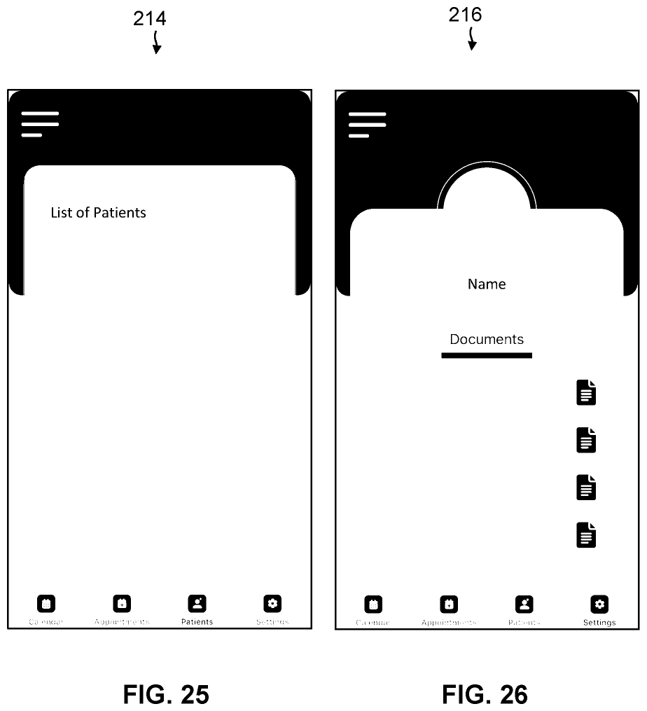

Referring to FIG. 25, the navigator style interface includes a patient's page 214. The patient list page 214 shows the health care professional a list of their patients.

Referring to FIG. 26, the navigator style interface includes a health care professional settings page 216. The health care professional settings page 216 shows the health care professional details included in the health care professional's profile, such as basic details (e.g., name, birth date, third party payment identifier, and address), account details (e.g., password, email address, and phone number), or document information (e.g., a link to or a copy of each of a CPSO Document, a CMPA document, an image of a front of a government-issued identification, and an image of a back of a government-issued identification).

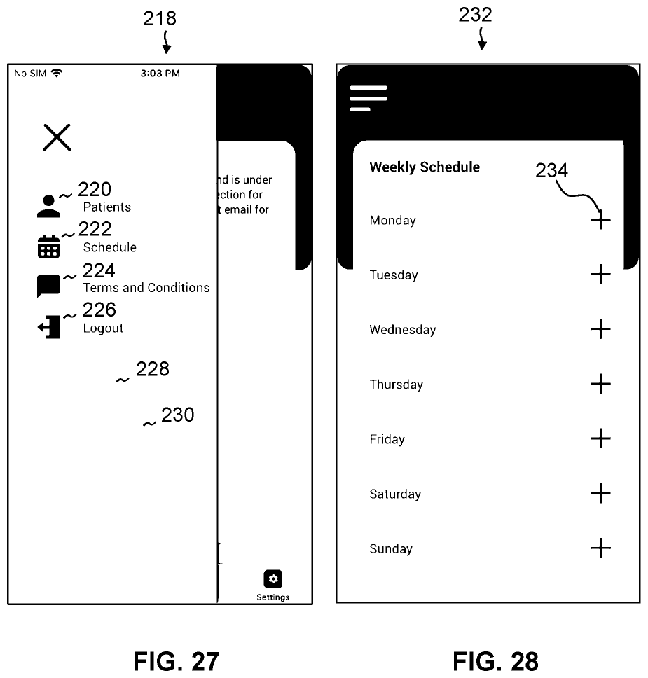

Referring to FIG. 27, the processor 104 may also be operable to present a health care professional menu page 218 via the navigator style interface on the health care professional device 120. The health care professional may be able to select, via the health care professional menu page 218, a patient's option 220 to return to the patient's page 214, a schedule option 222 to be taken to a schedule page 232 (FIG. 28), a terms and conditions option 224 to view terms and conditions, a logout option 226 to log out, a privacy policy option 228 to view a privacy policy, or a support and Frequently Asked Questions option 230 to view supporting information.

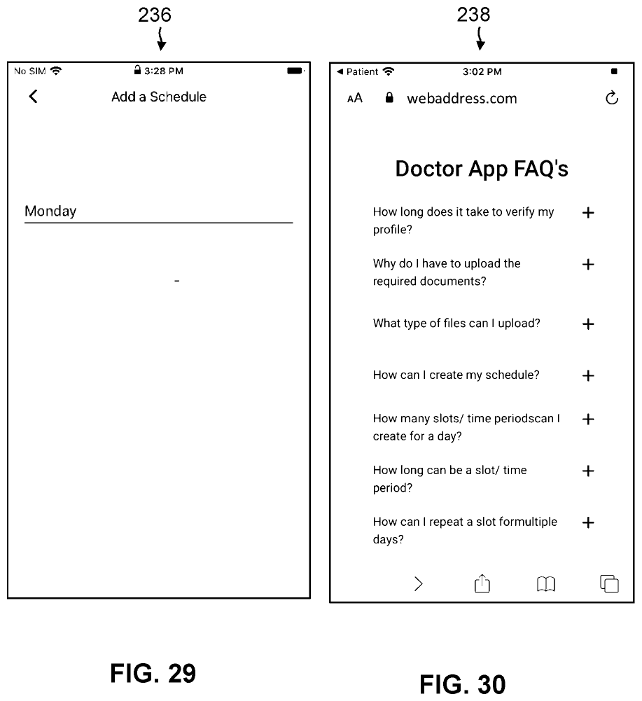

Referring to FIGS. 28 and 29, the schedule page 232 may allow a health care professional to set up available appointment slots. The system 100 may allow the health care professional a great deal of flexibility and control over their schedule. For example, the health care professional may select a Monday option 234 and be taken to an appointment set up page 236 to set up one or more appointments for the Monday. The health care professional may be able to create one or more appointment slots via the appointments set up page 236 of the navigator style interface on the health care professional device 120. For example, the health care professional may set up a first appointment slot from 9:00 am to 10:00 am, and a second appointment slot from 12:00 pm to 1:00 pm. The health care professional device 120 may be able to set up custom appointments (e.g., the health care professional is not limited to choosing from preset appointments). Accordingly, the health care professional may have some flexibility in choosing the start and end times of each appointment. The health care professional device 120 may be able to set a start time of an appointment at any one-minute increment (e.g., 12:01 pm or 12:29 pm rather than restricted to starting on the hour or half hour) and the appointment may automatically be set to be a predetermined length (e.g., 15 minutes) and/or the health care professional may similarly be able to set an end time of the appointment at any one-minute increment. However, for improved system functionality, the health care professional may be restricted to setting a start time on predetermined range of available start times, such as choosing from one of one the hour, quarter past, half past, and 45 minutes past the hour. Once a health care professional device 120 has set a slot or set of slots on a day, the health care professional device 120 may be used to repeat the slot or set of slots on one or more other days. In some examples, the health care professional device 120 is limited to entering a maximum number of slots per day for a health care professional (e.g., no more than 3 slots, which may be broken down automatically into short appointments such as appointments of no more than 15 minutes each).

In some examples, a health care professional device 120 may also be used to cancel one or more slots. However, if a slot is booked already, the at least one processor 104 may be operable to generate and display, via the navigator style interface on the health care professional device 120, a notice that the slot cannot be cancelled and/or must be cancelled in another way (e.g., that the health care professional must reach out to an administrator and/or the patient to cancel).

Referring now to FIG. 30, if the health care professional selects the support and Frequently Asked Questions option 230 the processor 104 is operable to generate a support and Frequently Asked Questions page 238 on the health care professional device 120. The terms and support and Frequently Asked Questions page 238 may show the health care professional a set of information (e.g., in the form of questions and associated answers).

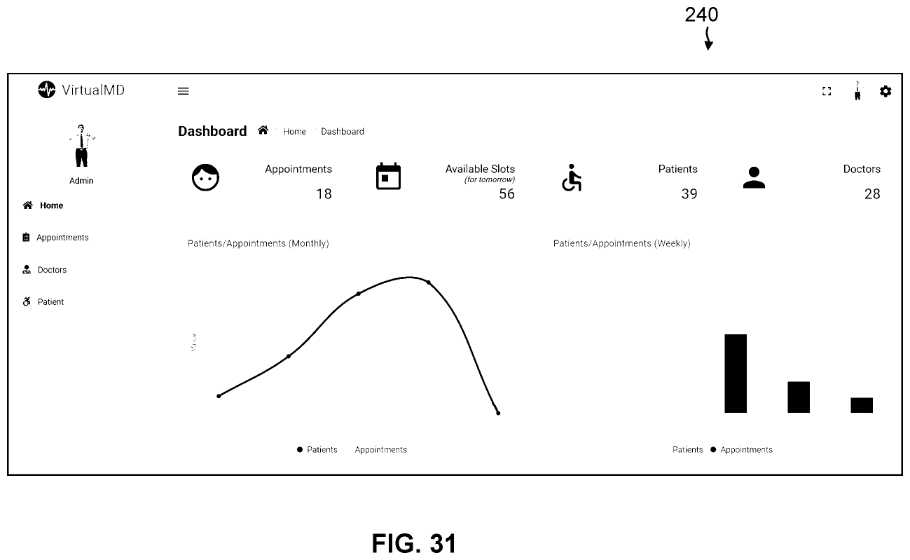

Referring now to FIG. 31, the processor 104 may be operable to generate a navigator style interface on the administrator device 119. For example, the navigator style interface on the administrator device 119 may include a home page 240. The home page 240 shows how many appointments have been booked, how many health care professionals are using the system 100, or how many patients are using the system 100.

The administrator device 119 may also, or alternatively, be able to perform one or more actions that a patient device 118 or health care professional device 120 may perform, such as updating a profile of a health care professional or patient. In some examples, the administrator device 119 may be used to generate and/or assign a health care professional and/or a patient to an appointment slot, and may be able to choose whether it is a basic appointment or includes one or more add-on services as discussed further below. The administrator device 119 may also be able to view details about one or more appointments regardless of which patient or health care professional is involved. In some examples, the administrator device 119 may be able to more an appointment as ‘Completed’ or ‘Cancelled’, as discussed further elsewhere herein.

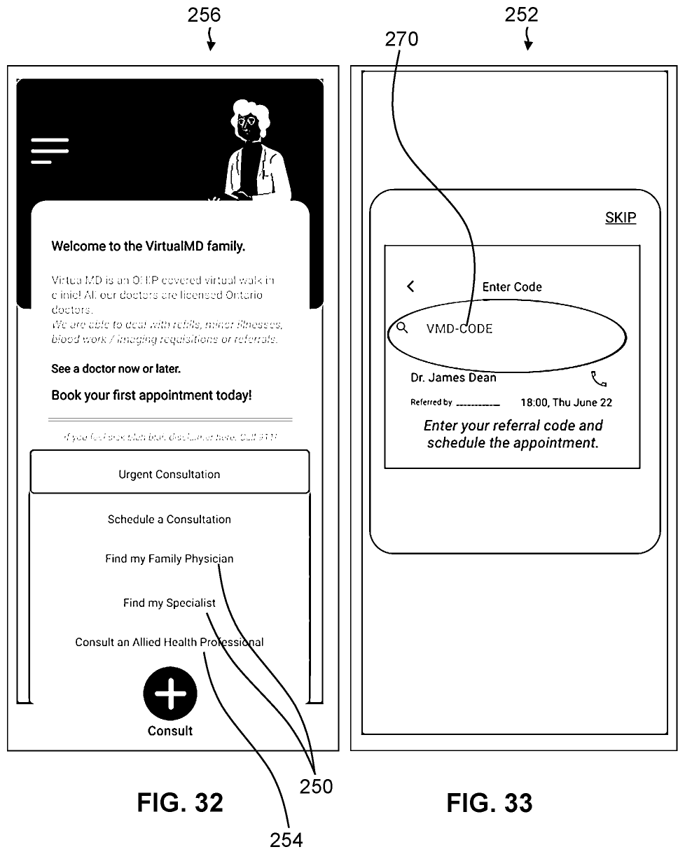

Referring now to FIGS. 32 and 33, in some examples the processor 104 is operable to present, on a patient communication device 118, via the navigator style interface, further options in addition to booking an urgent care appointment or a routine appointment. The processor 104 may be operable to present a third booking option 250 to find a particular health care provider, such as a patient's personal family doctor or a particular specialist. In some examples, if the patent knows a unique identifier for a particular health care provider (e.g., an identification code given to them by their family doctor or in a referral notice), they may be able to book an appointment with that health care provider by selecting the third booking option 250 (e.g., either for a family doctor or a specialist). The patient is then presented with an identifier-entry page 252 by the processor 104, and is able to enter their identifier (e.g., identifier 270) to be provided with access to book an appointment with that particular health care provider. For example, a doctor may give the patients of their own practice the doctor's identifier so that the patients can use the booking features for convenience, or a family doctor may make a referral and then the specialist's identifier may be provided to the patient. In some examples, the processor 104 is configured to prevent a general search for a family doctor or specialist doctor (e.g., a search by name or region), such as to promote the use of the random-assignment options 132, 134.

The patient may also be presented with a fourth booking option 254 to find an allied health care professional. The patient may be able to use the further booking option 254 to find, e.g., a psychologist, chiropractor, or physiotherapist.

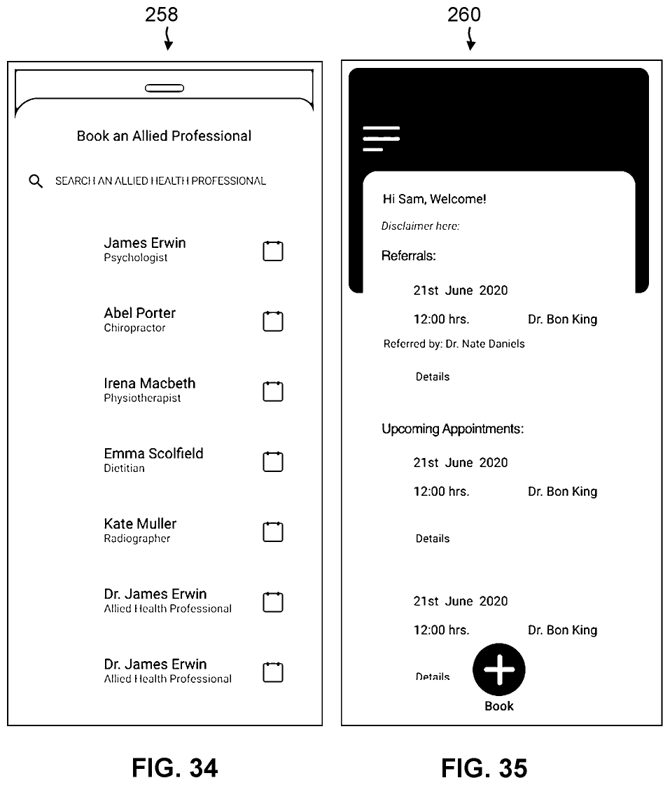

Referring now to FIG. 34, choosing the fourth booking option 254 (e.g., selecting the soft button on the options page 256 of FIG. 32) brings the patient to a search page 258. The patient may then search among the available allied health professionals for their preferred professional. Unlike for family doctors or specialist doctors, the patient may be able to search generally by keyword or other filter (e.g., rather than only accepting a random assignment or using an identifier known to the patient). Since many allied health care services may be paid services (i.e., not covered by a third party payor such as the Ontario health plan), the patient may be provided with more options for finding their preferred provider.

Referring now to FIG. 35, the processor 104 may also be able to provide the patient with further information in a summary page 260. The summary page 260 may show, and allow a patient to access, upcoming appointments. If the patent has been referred to another healthcare provider (e.g., a specialist or an allied professional), the summary page 260 may also show the referral. The patient may be able to access the referral to retrieve information about the referral. For example, the patient may need to access a health care provider identifier 270 (e.g., an ID number or code; FIG. 33) for the health care professional they have been referred to. As mentioned above, such an identifier may be used to search for the associated health care provider and book an appointment with them.

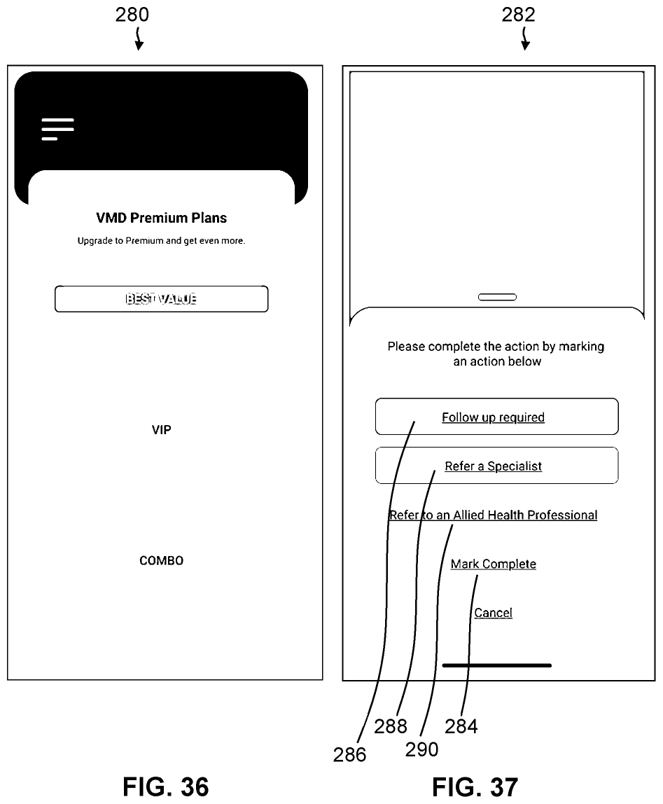

Referring now to FIG. 36, in some examples, a health care professional may pay to join the system. For example, the health care professional may pay a monthly fee to be available via the navigator style interface to patients for appointment booking. The health care professional may then be able to provide their patients with the health care provider's identifier so that the patients can find the health care provider and book appointments. In some examples, the processor 104 is operable to present, on a health care professional device via the navigator style interface, a sign up page 280. The health care provider may be able to use the sign up page 280 to choose a payment plan.

Referring now to FIG. 37, once a health care provider has joined the system and comes to the end of an appointment, in some examples they are presented with a plurality of closing options. For example, as depicted on the options page 282, the health care professional may be able to simply mark the appointment as complete via a “mark complete” option 284. However, in some examples, the health care professional may also be presented, via the navigator style interface, with a “follow up needed” option 286, a specialist referral option 288, and/or an allied health professional referral option 290.

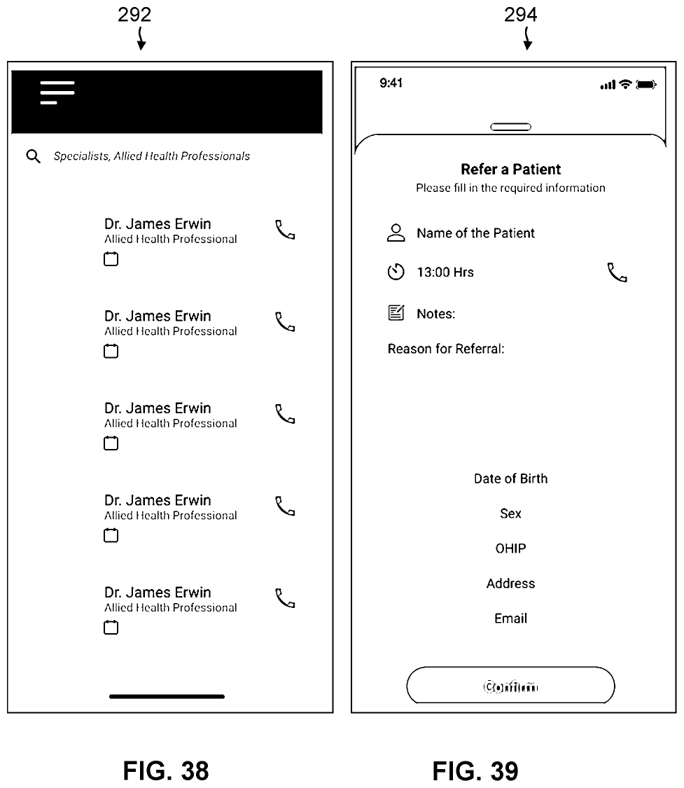

Referring now to FIGS. 38 and 39, where a referral option (e.g., option 288 or 290) is chosen, the health care provider may be provided with a referral selection page 292 via which the health care professional may select another health care professional to refer the patient who is associated with the finished appointment to. Once the health care professional to refer the patient to is selected, the first health care professional is presented with a details page 294 in which to add information about the patient, such as reasons for the referral.

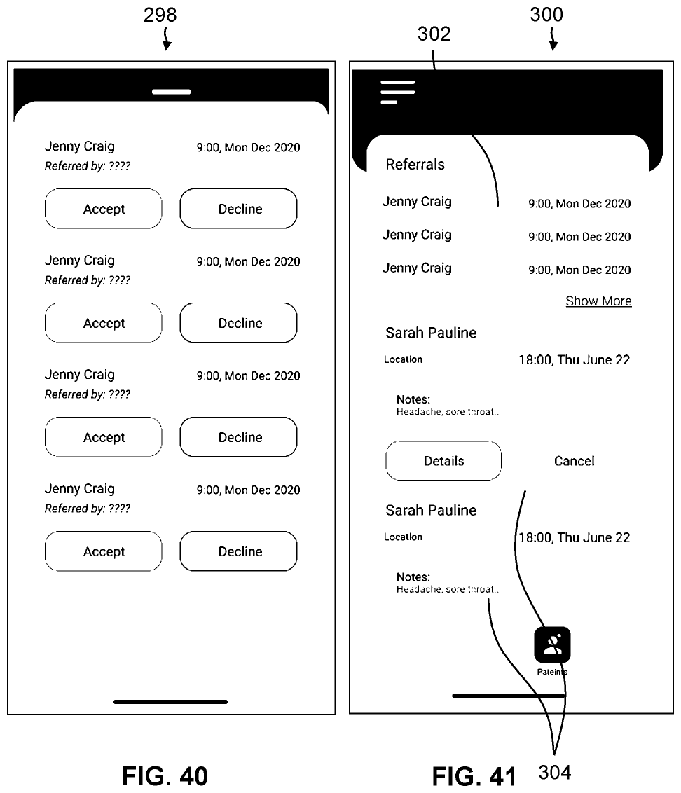

Referring now to FIGS. 40 and 41, in some examples, when a health care professional receives a referral the health care professional is able to choose whether to accept or decline the referral. The acceptance of the referral may be required before the identifier of the health care professional is released to the patient so that the patient is able to find and book and appointment with the health care professional. For example, the health care professional may be presented with an acceptance page 298. The health care professional may choose to view and either accept or decline a referral. Details of the referral 302 may also be added to the health care professional's patient summary page 300 if the health care professional accepts the referral, along with details of prior and/or upcoming appointments 304.

The present invention has been described here by way of example only. Various modification and variations may be made to these exemplary embodiments without departing from the scope of the invention, which is limited only by the appended claims.

1. A booking system, comprising:
a. at least one processor; and
b. at least one data storage device communicatively coupled to the at least one processor, and
c. wherein the at least one processor is operable to:
i. receive a referral request from a family doctor electronic communication device of a family doctor requesting to refer a patient to a specialist, the referral request including at least one criteria,
ii. identify a plurality of specialist doctor profiles stored on the at least one data storage device and matching the at least one criteria, and
iii. send a specialist notification to the family doctor electronic communication device for each of the plurality of specialist doctor profiles, each specialist notification including information from the associated specialist doctor profile.

2. The booking system of claim 1, wherein the at least one processor is further configured to
i. receive, in response to sending the notifications, a selection from the family doctor electronic communication device of a first specialist doctor profile of the plurality of specialist doctor profiles; and
ii. send a patient notice to a specialist doctor electronic communication device associated with the first specialist doctor profile, the patient notice including patient information from a patient profile of the patient stored on the at least one data storage device.

3. The booking system of claim 2, wherein the at least one processor is further configured to generate a referral notice and present the referral notice to the patient, wherein the referral notice includes information from the first specialist doctor profile.

4. A booking system, comprising:
a. at least one processor; and
b. at least one data storage device communicatively coupled to the at least one processor, and
c. wherein the at least one processor is operable to:
i. receive a request from a patient electronic communication device of a patient for an urgent care appointment,
ii. identify a next-available appointment slot in a plurality of doctor schedules stored on the at least one data storage device,
iii. book the next-available appointment slot as the urgent care appointment for the first patient, and
iv. provide a set of details of the next-available appointment to the patient electronic communication device.

5. The booking system of claim 4, wherein the set of details of the next-available appointment are a date and a time of day of the next-available appointment.

6. The booking system of claim 4, wherein the set of details includes an indication that the patient will receive a phone call at the date and time of day from the first doctor.

7. The booking system of claim 4, wherein the at least one processor is further configured, following (iii), to notify, via the navigator style interface on a doctor electronic communications device, a doctor associated with the next-available appointment slot that the next-available appointment slot has been booked.

8. A booking system, comprising:
a. at least one processor; and
b. at least one data storage device communicatively coupled to the at least one processor, and
c. wherein the at least one processor is operable to:
i. generate a navigator style interface on a doctor electronic communication device of a first doctor,
ii. prompt, via the navigator style interface on the doctor electronic communication device, the first doctor to provide a first credential,
iii. prompt, via the navigator style interface on the doctor electronic communications device, the first doctor to provide a first doctor schedule, the first doctor schedule including at least one available appointment slot,
iv. receive the first credential and the first doctor schedule from the doctor electronic communication device,
v. incorporate the first credential and the first doctor schedule into a first doctor profile stored on the at least one data storage device,
vi. generate the navigator style interface on an administrator electronic communication device of an administrator user,
vii. prompt, via the navigator style interface on the administrator electronic communication device, the administrator user to review the first credential,
viii. receive an approval of the first doctor profile from the administrator electronic communication device,
ix. mark the first doctor profile as an approved profile on the at least one data storage device,
x. generate the navigator style interface on a patient electronic communication device of a first patient,
xi. present, via the navigator style interface on the patient electronic communication device, the first patient with a first booking option to book an urgent care appointment,
xii. receive, following (xi), a first option response from the patient electronic communication device requesting the urgent care appointment,
xiii. identify, following (ix), a next-available appointment slot in the first doctor schedule on the at least one data storage device,
xiv. book the next-available appointment slot for the first patient in the first doctor schedule on the at least one data storage device, and
xv. present, via the navigator style interface on the patient electronic communication device, the first patient with a set of details of the next-available appointment.

9. The booking system of claim 8, wherein the at least one processor is further operable to prompt, via the navigator style interface on the doctor electric communications device, the first doctor to provide a first doctor government-issued identification, and wherein the approval from the administrator electronic communications device is received after prompting, via the navigator style interface on the administrator electronic communication device, the administrator user to review the first doctor government-issued identification.

10. The booking system of claim 9, wherein the first doctor government-issued identification includes a first image of a front side of an identification card and a second image of a rear side of the identification card.

11. The booking system of claim 8, wherein the first credential includes a document issued by a third party doctor regulator.

12. The booking system of claim 8, wherein the first credential includes at least one document issued by each of at least two third party doctor regulators.

13. The booking system of claim 8, wherein the set of details of the next-available appointment are a date and a time of day of the next-available appointment.

14. The booking system of claim 8, wherein the set of details includes an indication that the patient will receive a phone call at the date and time of day from the first doctor.

15. The booking system of claim 8, wherein the at least one processor is further configured, following (xiv), to notify, via the navigator style interface on the doctor electronic communications device, the first doctor that the first doctor schedule on the at least one data storage device, has been updated.

16. The booking system of claim 8, wherein the at least one processor is further configured, prior to (xi), to:
prompt, via the navigator style interface on the patient electronic communication device, the first patient to provide a first patient payment identifier;
receive the first patient payment identifier from the first patient electronic communication device; and
incorporate the first patient payment identifier into a first patient profile stored on the at least one data storage device.

17. The booking system of claim 16, wherein the first patient payment identifier is a third party identifier of the first patient issued by a third party payee or a set of personal payment information of the first patient.

18. The booking system of claim 8, wherein the at least one processor is operable to perform (xv) within one second of (xii).

19. The booking system of claim 18, wherein the set of details are presented via the navigator style interface on the patient electronic communication device as human-readable text.

20. A booking system, comprising:
a. at least one processor; and
b. at least one data storage device communicatively coupled to the at least one processor, wherein the at least one data storage device stores at least one patient profile associated with at least one patient and at least one schedule associated with at least one doctor, and
c. wherein the at least one processor is operable to:
i. generate a navigator style interface on a patient electronic communication device of a first patient of the at least one patient,
ii. present, via the navigator style interface on the patient electronic communication device, the first patient with a first booking option to book an urgent care appointment,
iii. receive, following (ii), a first option response from the patient electronic communication device requesting the urgent care appointment,
iv. identify a next-available appointment slot in the at least one schedule on the data storage device, wherein the next-available appointment slot is in a first schedule of the at least one schedule, wherein the first schedule is associated with a first doctor of the at least one doctor,
v. book the next-available appointment slot for the first patient,
vi. identify a first patient profile of the at least one patient profile on the at least one data storage device, the first patient profile associated with the first patient,
vii. send, send following (vi), the first patient profile to a first doctor electronic communication device of the first doctor, and
viii. present, via the navigator style interface on the patient electronic communication device, the first patient with a set of details of the next-available appointment.
